(19) United States
(12) Patent Application Publication
(10) Pub. No.: US 2002/0151041 A1
(43) Pub. Date: Oct. 17, 2002

(54) Enhancing surfaces for analyte detection

(76) Inventors: David I. Kreimer, Berkeley, CA (US); Thomas H. Nufert, Walnut Creek, CA (US)

Correspondence Address: Sheldon R. Meyer, Esq., FLIESLER DUBB MEYER & LOVEJOY LLP, Fourth Floor, Four Embarcadero Center, San Francisco, CA, 94111-4156, US

(21) Appl. No.: 10/099,675

(22) Filed: Mar. 15, 2002

Related U.S. Application Data
(60) Provisional application No. 60/276,197, filed on Mar. 15, 2001.

(51) Int. Cl. C12M 1/34; G01N 33/543

(52) U.S. Cl. 435/287.2; 436/518

(57) ABSTRACT

This invention comprises devices and methods for increasing the sensitivity of detection of analytes. Analytes are placed near enhancing structures on a substrate. The enhancing structures include fractal particle aggregates made of metals such as gold and silver. Analytes can be concentrated near enhancing structures using methods including spin-concentration, electroconcentration, and/or affinity methods. Affinity methods include hydrophobic (non-polar) interaction and hydrophilic (polar) interaction. By selecting appropriate substrate, enhancing structure, solvent and analyte, the devices and methods of this invention can increase the sensitivity of detection and quantitation of analytes using electromagnetic spectroscopy, including Raman spectroscopy.

### RELATED CASE

This application claims priority to United States Provisional Patent Application Serial No.: 60/276,197, filed Mar.15, 2001. This application is incorporated herein fully by reference.

### BACKGROUND

1. Field of the Invention

This invention relates to devices and methods for analyte detection. Specifically, the invention relates to devices and methods for enhancing signals generated by analytes. More specifically, the invention relates to devices and methods for the detection of analytes using Raman spectroscopy.

2. Description of Related Art

Detection of analytes of biological, technological or environmental interest is a task of great economic importance. Characterization of virtually any process can involve knowledge of the types and amounts of substances. The detection and quantification of molecules or “analytes” in complex mixtures containing small amounts of analyte and large numbers and amounts of other materials is a continuing challenge. As more interest is focused upon the roles of biological molecules in physiology and disease processes, the rapid accurate detection of biological molecules such as nucleic acids, proteins and low molecular weight materials is becoming more important.

I. Detection of Analytes

The detection of analyte, or “ligand” molecules is an important aspect of current biology, biotechnology, chemistry, and environmental industries. Detection of ligands can be accomplished using many different methods, including the chemical methods of chromatography, mass spectroscopy, nucleic acid hybridization and immunology.

For example, in biology and biotechnology industries, analytes such as deoxyribonucleic acid (“DNA”) and messenger ribonucleic acid (“mRNA”) are important indicators of specific genetic, physiological or pathological conditions. DNA can contain important information about the genetic makeup of an organism, and mRNA can be an important indicator of which genes are active in a specific physiological or pathological condition and what proteins may be created as a result of gene activation. Additionally, the direct detection of proteins can be important to the understanding of the physiological or pathological condition of an individual.

Currently available methods for the detection of nucleic acids, proteins and small molecular weight substances have undesirable characteristics. The methods are time consuming, require expensive equipment and reagents, require expert manual operations, and the reagents can be environmentally hazardous. Additionally, for assaying mRNA, the methods also can be sensitive to defects in the fidelity of reverse transcription. Unless the cDNA made during reverse transcription is exactly complementary to the mRNA, the analyte will not have the same sequence as the native mRNA, and misleading results can be obtained. The amplification of nucleic acid sequences by the polymerase chain reaction (“PCR”) has been used to increase the numbers of nucleic acid molecules (complementary DNA or “cDNA”) that can be detected.

Additionally, for medical diagnostic or forensic purposes, it can be very important for results of tests to be available rapidly. Commonly used methods for detection of specific nucleic acid sequences, proteins and small molecules can be too slow for therapeutic or forensic uses. Thus, there is a need for rapid, accurate measurement of analytes.

II. Raman Spectroscopy

Raman spectroscopy involves the use of electromagnetic radiation to generate a signal in an analyte molecule. Raman spectroscopic methods have only recently been developed to the point where necessary sensitivity is possible. Raman spectroscopic methods and some ways of increasing the sensitivity of Raman spectroscopy are described herein below.

A. Raman Scattering

According to a theory of Raman scattering, when incident photons having wavelengths in the near infrared, visible or ultraviolet range illuminate a certain molecule, a photon of that incident light can be scattered by the molecule, thereby altering the vibrational state of the molecule to a higher or a lower level. The vibrational state of a molecule is characterized by a certain type of stretching, bending, or flexing of the molecular bonds. The molecule can then spontaneously return to its original vibrational state. When the molecule returns to its original vibrational state, it can emit a characteristic photon having the same wavelength as the incident photon. The photon can be emitted in any direction relative to the molecule. This phenomenon is termed “Raleigh Light Scattering.”

A molecule having an altered vibrational state can return to a vibrational state different from the original state after emission of a photon. If a molecule returns to a state different from the original state, the emitted photon can have a wavelength different from that of the incident light. This type of emission is known as “Raman Scattering” named after C. V. Raman, the discoverer of this effect. If, a molecule returns to a higher vibrational level than the original vibrational state, the energy of the emitted photon will be lower (i.e., have longer wavelength) than the wavelength of the incident photon. This type of Raman scattering is termed “Stokes-shifted Raman scattering.” Conversely, if a molecule is in a higher vibrational state, upon return to the original vibrational state, the emitted photon has a lower energy (i.e., have a shorter wavelength). This type of Raman scattering is termed “anti-Stokes-shifted Raman scattering.” Because many more molecules are in the original state than in an elevated vibrational energy state, typically the Stokes-shifted Raman scattering will predominate over the anti-Stokes-shifted Raman scattering. As a result, the typical shifts of wavelength observed in Raman spectroscopy are to longer wavelengths. Both Stokes and anti-Stokes shifts can be quantitized using a Raman spectrometer.

B. Resonance Raman Scattering

When the wavelength of the incident light is at or near the frequency of maximum absorption for that molecule, absorption of a photon can elevate both the electrical and vibrational states of the molecule. The efficiency of Raman scattering of these wavelengths can be increased by as much as about 10⁸ times the efficiency of wavelengths substantially different from the wavelength of the absorption maximum. Therefore, upon emission of the photon with return to the ground electrical state, the intensity of Raman scattering can be increased by a similar factor.

C. Surface Enhanced Raman Scattering

When Raman active molecules are excited near to certain types of metal surfaces, a significant increase in the intensity of the Raman scattering can be observed. The increased Raman scattering observed at these wavelengths is herein termed “surface enhanced Raman scattering.” The metal surfaces that exhibit the largest increase in Raman intensity comprise minute or nanoscale rough surfaces, typically coated with minute metal particles. For example, nanoscale particles such as metal colloids can increase intensity of Raman scattering to about 10⁶ times or greater, than the intensity of Raman scattering in the absence of metal particles. This effect of increased intensity of Raman scattering is termed “surface enhanced Raman scattering.”

The mechanism of surface enhanced Raman scattering is not known with certainty,but one factor can affect the enhancement. Electrons can typically exhibit a vibrational motion, termed herein “plasmon” vibration. Particles having diameters of about 1/10 th the wavelength of the incident light can contribute to the effect. Incident photons can induce a field across the particles, and thereby can alter the movement of mobile electrons in the metal. As the incident light cycles through its wavelength, the induced motion of electrons can follow the light cycles, thereby creating an oscillation of the electron within the metal surface having the same frequency as the incident light. The electrons' motion can produce a mobile electrical dipole within the metal particle. When the metal particles have certain configurations, incident light can cause groups of surface electrons to oscillate in a coordinated fashion, thereby causing constructive interference of the electrical field so generated, creating an area herein termed a “resonance domain.” The enhanced electric field due to such resonance domains therefore can increase the intensity of Raman scattering and thereby can increase the intensity of the signal detected by a Raman spectrometer.

The combined effects of surface enhancement and resonance on Raman scattering is termed “surface enhanced resonance Raman scattering.” The combined effect of surface enhanced resonance Raman scattering can increase the intensity of Raman scattering by about 10¹⁴ or more. It should be noted that the above theories for enhanced Raman scattering may not be the only theories to account for the effect. Other theories may account for the increased intensity of Raman scattering under these conditions.

D. Raman Methods for Detection of Nucleic Acids and Proteins

Several methods have been used for the detection of nucleic acids and proteins. Typically, an analyte molecule can have a reporter group added to it to increase the ability of an analytical method to detect that molecule. Reporter groups can be radioactive, flourescent, spin labeled, and can be incorporated into the analyte during synthesis. For example, reporter groups can be introduced into cDNA made from mRNA by synthesizing the DNA from precursors containing the reporter groups of interest. Additionally, other types of labels, such as rhodamine or ethidium bromide can intercalate between strands of bound nucleic acids in the assay and serve as reporter groups of hybridized nucleic acid oligomers.

In addition to the above methods, several methods have been used to detect nucleic acids using Raman spectroscopy. Vo-Dinh, U.S. Pat. No: 5,814,516; Vo-Dinh, U.S. Pat. No: 5,783,389; Vo-Dinh, U.S. Pat. No: 5,721,102; Vo-Dinh, U.S. Pat. No: 5,306,403. These patents are herein incorporated fully by reference. Recently, Raman spectroscopy has been used to detect proteins. Tarcha et al., U.S. Pat. No: 5,266,498; Tarcha et al., U.S. Pat. No: 5,567,628, both incorporated herein fully by reference, provide an analyte that has been labeled using a Raman active label and an unlabeled analyte in the test mixture. The above-described methods rely upon the introduction of a Raman active label, or “reporter” group, into the analyte molecule. The reporter group is selected to provide a Raman signal that is used to detect and quantify the presence of the analyte.

By requiring reporter groups to be introduced into the analyte, additional steps and time are required. Additionally, the above methods can require extensive washing of the bound and unbound Raman labeled analytes to provide the selectivity and sensitivity of the assay. Moreover, because specific Raman labels must be provided for each type of assay system used, properties of the analytes must be determined in advance of the assay.

### SUMMARY OF THE INVENTION

This invention comprises devices for improving the detection of analytes. The devices and methods can provide localization of an analyte to an area near an enhancing structure, such as a fractal aggregate.

In certain embodiments, a solution is applied to the surface of a device, and a portion of the solution is in contact with an enhancing structure. The analytes that are near the enhancing structure can exhibit greater signal than analytes at more distant sites from an enhancing structure.

In other embodiments, the device can comprise a porous substrate having an area with enhancing structures thereon. The pores in the substrate can be sufficiently small so that analytes do not pass through the pores, but solvents can, thus concentrating the analyte near the enhancing structures. In certain of these embodiments, a sample is placed on the substrate and the solution is drawn through the porous substrate by a hydrostatic or osmotic pressure gradient.

In yet other embodiments, a substrate need not be porous, but rather can have a hydrophobicity different from that of the solution in which the analyte is present. For example, if a substrate is hydrophobic relative to water, an organic analyte tends to adsorb onto the hydrophobic substrate near the fractal, enhancing structures. Conversely, one can use reverse-phase to concentrate relatively hydrophilic analytes within relatively hydrophobic media near enhancing structures.

In alternative embodiments, the invention comprises an electrode having enhancing structures thereon. An electrical field can be used to attract a charged analyte to the surface, near the enhancing structure, thereby increasing the magnitude of an electromagnetic, or other signal characteristic of the analyte.

In yet other embodiments, analytes can adhere to a surface and enhancing structures can be applied on top of the analytes and the surface.

### BRIEF DESCRIPTION OF THE FIGURES

The invention will be described with respect to the particular embodiments thereof. Other objects, features, and advantages of the invention will become apparent with reference to the specification and drawings in which:

FIGS. 1a-1b depict an enhancing surface of this invention in the form of a cuvette. FIG. 1a depicts a cross-sectional view and FIG. 1b depicts a top view. FIGS. 2a and 2b depict two embodiments of enhancing surfaces of this invention.

FIGS. 3a-3b depict embodiments of this invention. FIG. 3a depicts a porous substrate having enhancing structures and analytes thereon. FIG. 3b depicts a cuvette of this invention, in which a porous substrata as in FIG. 3a is positioned within a tube.

FIG. 4 depicts an alternative embodiment of this invention in which an enhancing surface is also an electrode.

FIGS. 5a-5c depict embodiments of this invention wherein an enhancing surface is hydrophobic.

FIGS. 6a-6c depict alternative embodiments of this invention wherein a hydrophobic analyte is concentrated near a hydrophobic enhancing structure.

FIGS. 7a-7b depict alternative embodiments of this invention wherein a hydrophobic analyte is concentrated near a hydrophobic enhancing structure.

FIGS. 8a-8b depict an alternative embodiment of this invention wherein a hydrophilic analyte is concentrated near a hydrophilic enhancing structure.

FIGS. 9a-9b depict an alternative embodiment in which an isoelectric focusing gel is applied to an enhancing surface. FIG. 9a depicts a gel after isoelectric focusing that is positioned over an enhancing surface. FIG. 9b depicts the substrate after transfer of the analytes from isoelectric focusing gel.

FIGS. 10a-10b depict an alternative embodiment in which analytes are placed on a substrate and enhancing structures are applied on the top thereof.

### DETAILED DESCRIPTION

Definitions

The following words and terms are used herein. The term “analyte” as used herein includes molecules, particles or other material whose presence and/or amount is to be determined. Examples of analytes include but are not limited to deoxyribonucleic acid (“DNA”), ribonucleic acid (“RNA”), amino acids, proteins, peptides, sugars, lipids, vitamins, co-factors, glycoproteins, cells, sub-cellular organelles, aggregations of cells, and other materials of biological interest.

The term “fractal” as used herein includes a structure comprised of elements, and having a relationship between the scale of observation and the number of elements, i.e., scale-invariant. By way of illustration only, a continuous line is a 1-dimensional object. A plane is a two-dimensional object and a volume is a three-dimensional object. However, if a line has gaps therein, and is not a continuous line, the dimension is less than one. For example, if ½ of the line is missing, then the fractal dimension is ½. Similarly, if points on a plane are missing, the fractal dimension of the plane is between one and 2. If ½ of the points on the plane are missing, the fractal dimension is 1.5. Moreover, if ½ of the points of a solid are missing, the fractal dimension is 2.5. In scale invariant structures, the structure of objects appears to be similar, regardless of the size of the area observed. Thus, fractal structures are a type of ordered structures, as distinguished from random structures, which are not ordered.

The term “fractal associate” as used herein, includes a structure of limited size, comprising at least about 100 individual particles associated together, and which demonstrates scale invariance within an area of observation limited on the lower bound by the size of the individual particles comprising the fractal associate and on the upper bound by the size of the fractal associate.

The term “fractal dimension” as used herein, means the exponent D of the following equation: N∝R^{D}, where R is the area of observation, N is the number of particles, and D is the fractal dimension. Thus in a non-fractal solid, if the radius of observation increases by 2-fold, the number of particles observed within the volume increases by 2³. However, in a corresponding fractal, if the radius of observation increases by 2-fold, the number of particles observed increases by less than 2³.

The term “fractal particle associates” as used herein includes a large number of particles arranged so that the number of particles per unit volume (the dependent variable) or per surface unit changes non-linearly with the scale of observation (the independent variable).

The term “label” as used herein includes a moiety having a physicochemical characteristic distinct from that of other moieties that permit determination of the presence and/or amount of an analyte of which the label is a part. Examples of labels include but are not limited to fluorescence, spin-resonance, radioactive moieties. Also known as reporter group.

The term “linker” as used herein includes an atom, molecule, moiety or molecular complex having two or more chemical groups capable of binding to a surface and permitting the attachment of particles together to form groups of particles. The simplest linker connects two particles. A branched linker may link together larger numbers of particles.

The term “ordered structures” as used herein includes structures that are non-random.

The term “particle structures” as used herein includes a group of individual particles that are associated with each other in such a fashion as to permit enhancement of electric fields in response to incident electromagnetic radiation. Examples of particles include metals, metal-coated polymers and fullerenes. Also included in the meaning of the term “particle structures” are films or composites comprising particles on a dielectric surface or imbedded in a dielectric material.

The term “percolation point” as used herein includes a point in time on a conductive surface or medium when the surface exhibits an increase in conductance, as measured either via surface or bulk conductance in the medium. One way to measure surface or “sheet” conductance is via electric probes applied to the surface.

The term “Raman signal” as used herein includes a Raman spectrum or portion of Raman spectrum.

The term “Raman spectral feature” as used herein includes a value obtained as a result of analysis of a Raman spectrum produced for an analyte under conditions of detection. Raman spectral features include, but are not limited to, Raman band frequency, Raman band intensity, Raman band width, a ratio of band widths, a ratio of band intensities, and/or combinations the above.

The term “Raman spectroscopy” as used herein includes a method for determining the relationship between intensity of scattered electromagnetic radiation as a function of the frequency of that electromagnetic radiation.

The term “Raman spectrum” as used herein includes the relationship between the intensity of scattered electromagnetic radiation as a function of the frequency ofthat radiation.

The term “random structures” as used herein includes structures that are neither ordered nor fractal. Random structures appear uniform regardless of the point and scale of observation, wherein the scale of observation encompasses at least a few particles.

The term “receptor” as used herein means a moiety that can bind to or can retain an analyte under conditions of detection.

The term “resonance” as used herein includes an interaction with either incident, scattered and/or emitted electromagnetic radiation and a surface having electrons that can be excited by the electromagnetic radiation and increase the strength of the electric field of the electromagnetic radiation.

The term “resonance domain” as used herein includes an area within or in proximity to a particle structure in which an increase in the electric field of incident electromagnetic radiation occurs.

The term “reporter group” as used herein includes label.

The term “scaling diameter” as used herein means a relationship between particles in a nested structure, wherein there is a ratio (scaling ratio) of particle diameters that is the same, regardless of the size of the particles.

The term “surface enhanced Raman spectroscopy” (“SERS”) as used herein includes an application of Raman spectroscopy in which intensity of Raman scattering is enhanced in the presence of an enhancing surface.

The term “surface enhanced resonance Raman spectroscopy” (“SERRS”) as used herein includes an application of Raman spectroscopy in which Raman signals of an analyte are enhanced in the presence of an enhancing surface (see SERS) and when an absorption band of the analyte overlaps with the wavelength of incident electromagnetic radiation.

Embodiments of the Invention

Detection of analytes is an important aim of research and development projects in many situations. Raman spectroscopy can provide a means for detecting and quantifying a variety of analytes without the need to label the analyte, and thus, can increase the speed and efficiency of detection and analysis. However, in general, signals generated by Raman scattering are relatively weak, and effective analysis can be improved by increasing the intensity of Raman signals. Thus, this invention includes materials and methods for increasing the amplitude of Raman signals generated by analytes.

Several of the embodiments of this invention involve devices, materials and methods for increasing the amplitude of Raman signals by positioning an analyte near an enhancing structure. By positioning analytes near enhancing structures, the intensity of SERS and SERRS signals can be increased.

I. Manufacture of Enhancing Structures

The Raman active structures desirable for use according to this invention can include any structure in which Raman signals can be amplified. The following discussion regarding metal fractal structures is not intended to be limiting to the scope of the invention, but is for purposes of illustration only.

A. Manufacture of Metal Particles

To make metal particles according to some embodiments of this invention, we can generally use methods known in the art. Tarcha et al., U.S. Pat. No: 5,567,628, incorporated herein fully by reference. Additional methods are described in co-pending U.S. patent application Ser. No: 09/670,453, filed Sep. 26, 2000, incorporated herein fully by reference. Metal colloids can be composed of noble metals, specifically, elemental gold or silver, copper, platinum, palladium and other metals known to provide surface enhancement. In general, to make a metal colloid, a dilute solution containing the metal salt is chemically reacted with a reducing agent. Reducing agents can include ascorbate, citrate, borohydride, hydrogen gas, and the like. Chemical reduction of the metal salt can produce elemental metal in solution, which combine to form a colloidal solution containing metal particles that are relatively spherical in shape.

### EXAMPLE 1

### Manufacture of Gold Colloid and Fractal Structures

In one embodiment of this invention, a solution of gold nuclei is made by preparing a 0.01% solution of NaAuCl₄ in water under vigorous stirring. One milliliter (“ml”) of a solution of 1% sodium citrate is added. After 1 minute of mixing, 1 ml of a solution containing 0.075% NaBH₄ and 1% sodium citrate is added under vigorous stirring. The reaction is permitted to proceed for 5 minutes to prepare the gold nuclei having an average diameter of about 2 nm). The solution containing the gold nuclei can be refrigerated at 4° C. until needed. This solution can be used as is, or can be used to produce particles of larger size (e.g., up to about 50 nm diameter), by rapidly adding 30 μl of the solution containing gold nuclei and 0.4 ml of a 1% sodium citrate solution to the solution of 1% HAuCl₄.₃H₂O diluted in 100 ml H₂O, under vigorous stirring. The mixture is boiled for 15 minutes and is then cooled to room temperature. During cooling, the particles in the solution can form fractal structures. The resulting colloid and/or fractal particle structures can be stored in a dark bottle.

Deposition of enhancing particles on dielectric surfaces including glass can generate films that can enhance electromagnetic signals. Such films can be as thin as about 10 nm. In particular, the distribution of electric field enhancement on the surface of such a film can be uneven. Such enhancing areas are resonance domains. Such areas can be particular useful for positioning receptors for analyte binding and detection. For films or particle structures embedded in dielectric materials, one way to manufacture enhancing structures is to treat the surface until “percolation points” appear. Methods for measuring sheet resistance and bulk resistance are well known in the art.

### EXAMPLE 2

### Manufacture of Metal Particles and Fractal Structures Using Laser Ablation

In addition to liquid phase synthesis described above, laser ablation is used to make metal particles. A piece of metal foil is placed in a chamber containing a low concentration of a noble gas such as helium, neon, argon, xenon, or krypton. Exposure to the foil to laser light or other heat source causes evaporation of the metal atoms, which, in suspension in the chamber, can spontaneously aggregate to form fractal or other particle structures as a result of random diffusion. These methods are well known in the art.

B. Manufacture of Films Containing Particles

To manufacture substrates containing metal colloidal particles of one embodiment of this invention, the colloidal metal particles can be deposited onto quartz slides as described in Examples 1 or 2. Other films can be made that incorporate random structures or non-fractal ordered structures in similar fashions.

### EXAMPLE 3

### Manufacture of Quartz Slides Containing Gold Fractal Structures

Quartz slides (2.5 cm×0.8 cm×0.1 cm) are cleaned in a mixture of HCl:HNO₃ (3:1) for several hours. The slides are then rinsed with deionized H₂O (Millipore Corporation) to a resistance of about 18 MΩ and then with CH₃OH. Slides are then immersed for 18 hours in a solution of aminopropyltrimethoxysilane diluted 1:5 in CH₃OH. The slides are then rinsed extensively with CH₃OH (spectrophotometric grade) and deionized H₂O prior to immersion into colloidal gold solution described above. The slides are then immersed in the gold colloid solution above. During this time, the gold colloid particles can deposit and can become attached to the surface of the quartz slide. After 24 hours, colloid derivatization is complete. Once attached, the binding of colloidal gold nanocomposites to the quartz surfaces is strong and is essentially irreversible. During the procedure, ultraviolet and/or visual light absorbance spectra of such derivatized slides are used to assess the quality and reproducibility of the derivatization procedure. The manufacturing process is monitored using electron microscopy to assess the density of the colloidal coating, the distribution of gold colloid particles on the surface, and the size of the gold colloid particles.

C. Aggregation of Particles to Form Particle Structures

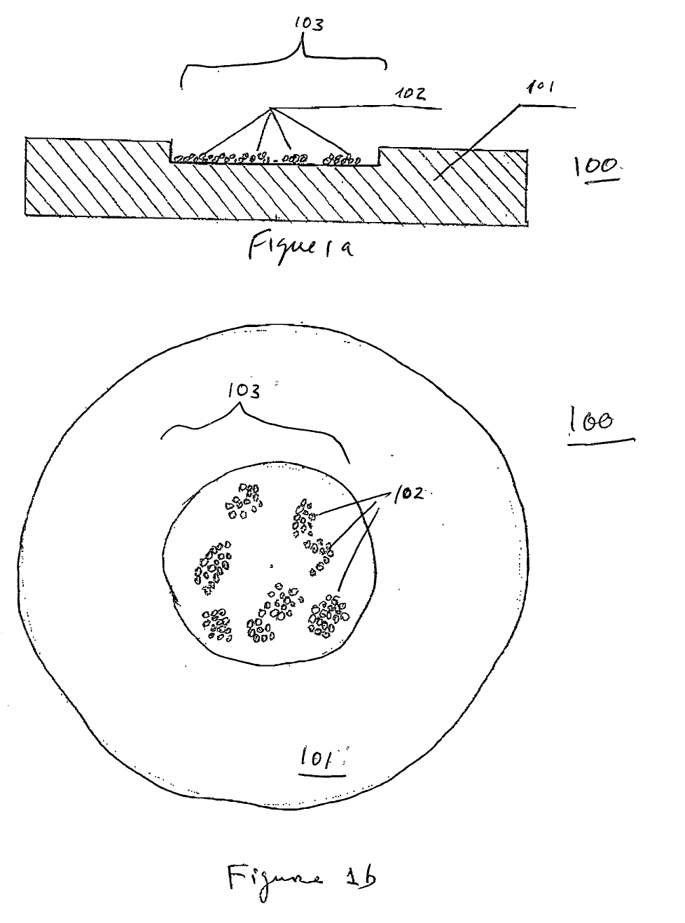

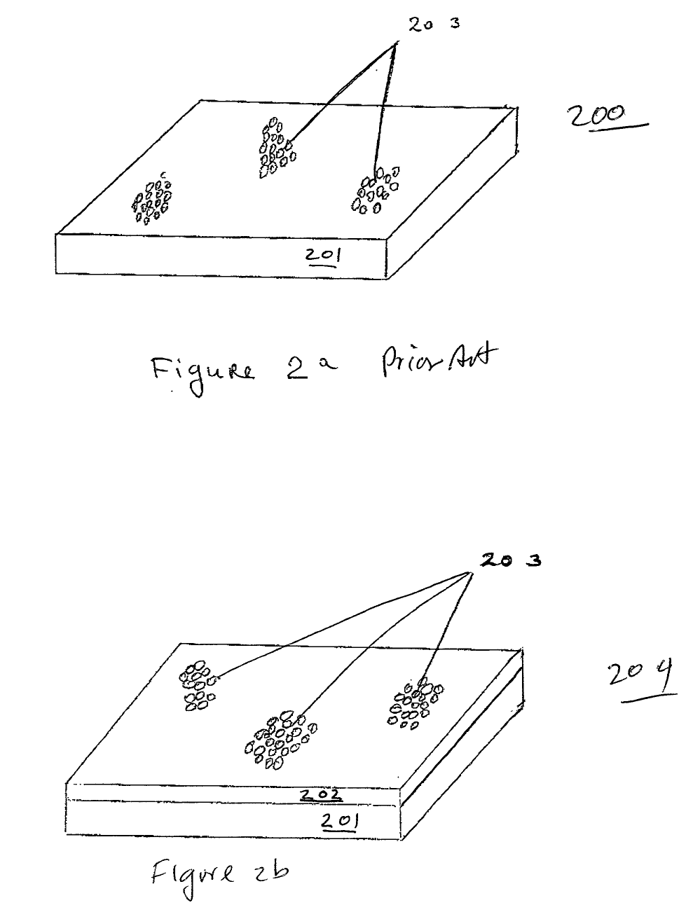

According to other embodiments of this invention, several methods can be used to form particle structures. It is known that metal colloids can be deposited onto surfaces, and when aggregated can form fractal structures having a fractal dimension of about 1.8. Safonov et al., Physical Review Letters 80(5):1102-1105 (1998 ) incorporated herein fully by reference. FIG. 1 depicts a particle structure suitable for use with the methods of this invention. The particles are arranged in a scale-invariant fashion, which promotes the formation of resonance domains upon illumination by laser light.

In addition to fractal structures, ordered non-fractal structures and random structures can be generated. These different types of structures can have desirable properties for enhancing signals associated with detection of analytes using electromagnetic radiation.

To make ordered non-fractal structures, one can use, for example, chemical linkers having different lengths sequentially as described in more detail below. In addition, using linkers of the same size, one can generate ordered structures, which can be useful for certain applications.

In certain embodiments of this invention, particles can be attached together to form structures having resonance properties. In general, it can be desirable to have the particles being spheres, ellipsoids, or rods. For ellipsoidal particles, it can be desirable for the particles to have a long axis (x), another axis (y) and a third axis (z). In general, it can be desirable to have x be from about 0.05 to about 1 times the wavelength (λ) of the incident electromagnetic radiation to be used. For rods, it can be desirable for x to be less than about 4λ, alternatively, less than about 3λ, alternatively less than about 2λ, in other embodiments, less than about 1λ, and in yet other embodiments, less than about l/2λ. The ends of the rods can be either flat, tapered, oblong, or have other shape that can promote resonance.

For two particle structures, it can be desirable for the particle pair to have an x dimension to be less than about 4λ, alternatively, less than about 3λ, alternatively less than about 2λ, in other embodiments, less than about 1λ, and in yet other embodiments, less than about ½λ.

For two-dimensional structures, pairs of particles, rods, rods plus particles together can be used. The arrangement of these elements can be randomly distributed, or can have a distribution density that is dependent upon the scale of observation in a non-linear fashion.

In other embodiments, rods can be linked together end-to end to form long structures that can provide enhanced resonance properties.

For three-dimensional structures, one can use regular nested particles, or chemical arrays of particles, associated either by chemical linkers in a fractal structure or in ordered, nested arrays.

In yet other embodiments, of third-order structures, a suspension of particles can be desirable. In certain of these embodiments, the suspended particles can have dimensions in the range of about ½λ to about 1 millimeter (mm).

Using the strategies of this invention, a researcher or developer can satisfy many needs, including, but not limited to selecting the absorbance of electromagnetic radiation by particle elements, the nature of the surface selected, the number of resonance domains, the resonance properties, the wavelengths of electromagnetic radiation showing resonance enhancement, the porosity of the particle structures, and the overall structure of the particle structures including, but not limited to the fractal dimensions of the structure(s).

### 1. Photoaggregation

Photoaggregation can be used to generate particle structures that have properties which can be desirable for use in Raman spectroscopy.

Irradiation of fractal metal nanocomposites by a laser pulse with an energy above a certain threshold leads to selective photomodification, a process that can result in the formation of “dichroic holes” in the absorption spectrum near the laser wavelength (Safonov et al., Physical Review Letters 80(5):1102-1105 (1998), incorporated herein fully by reference). Selective photomodification of the geometrical structure can be observed for both silver and gold colloids, polymers doped with metal aggregates, and films produced by laser evaporation of metal targets.

One theory for the formation of selective photomodification is that the localization of optical excitations in fractal structures are prevalent in random nanocomposites. According to this theory, the localization of selective photomodification in fractals can arise because of the scale-invariant distribution of highly polarizable particles (monomers). As a result, small groups of particles having different local configurations can interact with the incident light independently of one another, and can resonate at different frequencies, generating different domains, called herein “optical modes.” According to the same theory, optical modes formed by the interactions between monomers in fractal are localized in domains that can be smaller than the optical wavelength of the incident light and smaller than the size of the clusters of particles in the colloid. The frequencies of the optical modes can span a spectral range broader than the absorption bandwidth of the monomers associated with plasmon resonance at the surface. However, other theories may account for the effects of photomodification of fractal structures, and this invention is not limited to any particular theory for operability.

Photomodification of silver fractal aggregates can occur within domains as small as about 24×24×48 nm³ (Safonov et al., Physical Review Letters 80(5):1102-1105 (1998), incorporated herein fully by reference). The energy absorbed by the fractal medium can be localized in a progressively smaller number of monomers as the laser wavelength is increased. As the energy absorbed into the resonant domains increases, the temperature at those locations can increase. At a power of 11 mJ/cm², light having a wavelength of 550 nm can produce a temperature of about 600 K (Safonov et al., Physical Review Letters 80(5):1102-1105 (1998), incorporated herein fully by reference). At this temperature, which is about one-half the melting temperature of silver, sintering of the colloids can occur (Safonov et al., Id.) incorporated herein fully by reference), thereby forming stable fractal nanocomposites.

As used in this invention, photoaggregation can be accomplished by exposing a metal colloid on a surface to pulses of incident light having a wavelengths in the range of about 400 nm to about 2000 nm. In alternative embodiments, the wavelength can be in the range of about 450 nm to about 1079 nm. The intensity of the incident light can be in the range of about 5 mJ/cm² to about 20 mJ/cm². In an alternative embodiment, the incident light can have a wavelength of 1079 nm at an intensity of 11 mJ/cm².

Fractal aggregates that are especially useful for the present invention can be made from metal particles having dimensions in the range of about 10 nm to about 100 nm in diameter, and in alternative embodiments, about 50 nm in diameter. A typical fractal structure of this invention is composed of up to about 1000 particles, and an area of the aggregate typically used for large-scale arrays can have a size of about 100 μm×100 μm.

FIG. 2 depicts a particle structure that have been photoaggregated and that are suitable for use with the methods of this invention. Local areas of fusion of the metal particles can be observed (circles).

II Surfaces Having Enhancing Structures

Surfaces having enhancing structures comprise one or more of a variety of different shaped materials and different types of materials. In certain embodiments, the surface can be quartz or quartz glass. The types of dielectric materials need not be limited to glass or other silicon dioxide type materials. Rather, organic substances, such as polyacrylamide, polystyrene, and the like can be suitable, so long as the material does not contribute substantially to the signal detected on the surface. In other embodiments, a layer of a metal, such as gold is applied to the surface of a dielectric material, and can provide desirable properties, which include masking Raman signals generated by the substrate and preventing unwanted adherence of analytes to the substrate. In both types of embodiments, enhancing structures can then be applied to the surface, where they adhere. In those embodiments, a the layer of metal can be deposited by a variety of methods known in the art.

The substrates can be planar, or alternatively can be in the form of a cuvette, in which a hollow tube or “well” has an enhancing surface therein. In several alternative embodiments, the enhancing material can be placed at the bottom of the cuvette. In other embodiments, an enhancing surface can be provided on sidewalls of the cuvette.

FIG. 1a depicts a cross-sectional view of an embodiment 100 of this invention in which substrate 101 has enhancing structures 102 within a well 103. FIG. 1b is a top view of an embodiment of the invention as in FIG. 1a.

FIG. 2a depicts a prior art surface 200 comprising a substrate 201 having enhancing structures 203 thereon. FIG. 2b depicts a surface of this invention wherein a layer of gold metal 202 is on the top of a substrate 201. Enhancing structures 203 are attached to gold surface 202.

B. Spin-Concentration Devices

In certain embodiments of this invention, analytes can be positioned selectively close to enhancing structures using a spin-concentrating device. Such devices include a tube and a porous membrane or disk positioned across the tube, thereby separating the tube into a top portion and a bottom portion. The tube can have any convenient shape, and can have a circular, square, rectangular, triangular, pentagonal, hexagonal or other cross-section, The top portion can be used to place a sample comprising an analyte of interest and a solvent compatible with the analyte. After placing the sample in the top portion, the spin-concentrating device can be placed in a centrifuge. The centrifuge is spun to sediment the analytes onto the surface having enhancing structures. In certain embodiments, the substrate can be porous so as to permit solvent and other non-analytes to pass through into a bottom portion of the spin-concentration device. In such embodiments, the analyte can be concentrated, in the absence of undesired molecules, near the enhancing structures.

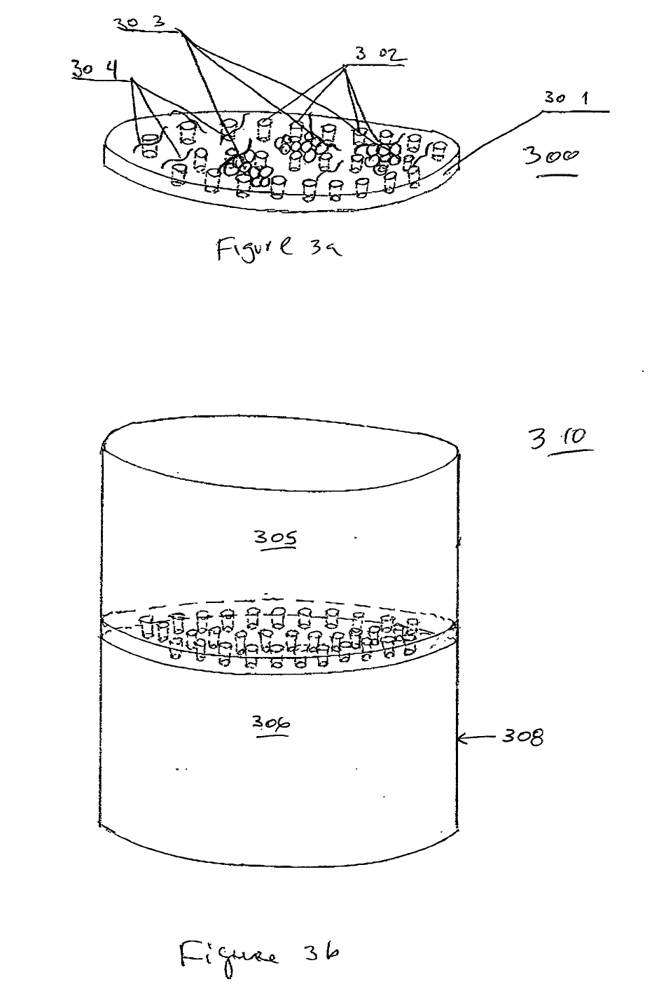

FIG. 3a depicts a porous substrate of this invention 300 after spin-concentration. Substrate 301 has pores 302 therethrough to permit passage of solvent and undesired molecules. Enhancing structures 303 are attached to substrate 301. Analyte molecules 304 were present in an original solution applied to the spin-concentration device. After solvent passes through the pores 302, the analytes are depicted close to enhancing structures 303.

FIG. 3b depicts an embodiment 310 of this invention having a porous substrate 301 as shown in FIG. 3a within tube 308. Porous substrate 301 is shown in tube 308 dividing the volume of tube 308 into a top reservoir 305 and a bottom portion 306. In use, enhancing structures can be attached to porous substrate 301. An analyte solution (not shown) is placed in top reservoir 305. A hydrostatic pressure difference between top reservoir 308 and bottom portion 306 is generated, for example, by gravity or by centrifugation. Liquid molecules are sufficiently small so as to pass through pores 302 in response to the hydrostatic pressure gradient. Analyte molecules 304 are too large to easily pass through pores 302, and therefore are retained on the surface of the substrate 301, and are positioned near enhancing structures 303.

### C. Electroconcentrating Devices

In other embodiments, charged analytes can be concentrated near enhancing structures by the application of electrical fields. A positively charged ion (“cation”) can be attracted to a negatively charged surface. Conversely, a negatively charged ion (“anion”) can be attracted to a positively charged surface. The charged analytes can thus be concentrated near enhancing structures on the charged surface.

FIG. 4 depicts an alternative embodiment 400 of this invention. An electroconcentration device has tube 401 has sealed holes 402 adapted to permit passage of wires 403 into the interior of tube 401. Wires 403 are connected to an electric power supply 404 which includes a switch. One wire is attached to substrate 406, which is an electrode and has enhancing structures 407 thereon. The other wire is attached to electrode 405. Incident beam of electromagnetic radiation 408 impinges on analytes near enhancing structures 407, and thereby generates an emitted beam of electromagnetic radiation 409, which can be detected by a detector (not shown).

In certain other embodiments, analytes can be concentrated in the absence of a liquid solvent. For airborne materials, the sample of air or other gas containing particles comprising analytes of interest can be introduced into tube 401. Application of positive charge to the electrode on substrate 406 can attract anionic analytes can be concentrated near electrode 406. Alternatively, to concentrate cationic analytes on enhancing surface 406, a similar procedure can be used, except that the substrate/electrode 406 should have a negative electric charge applied thereto.

### D. Affinity Interaction

Other embodiments can use other physicochemical principles for concentrating analytes. Alternatives include affinity interactions including hydrophobic interaction and hydrophilic interaction principles. In hydrophobic interaction, a lipophyllic (or hydrophobic) molecule is applied to a lipophyllic substrate. IN certain embodiments, the hydrophobic molecule can be dissolved in a polar solvent, such as water or an alcohol. A lipophyllic molecule tends to partition between the lipophyllic surface and the polar solvent phase. Due to the inherent motion of molecules in solution (“Brownian motion”), certain of the lipophyllic molecules will move to the lipophyllic substrate. Conversely, certain of the lipophyllic molecules in solution will move off of the lipophyllic substrate and into the polar phase. However, until an equilibrium is reached, the overall numbers of lipophyllic molecules moving onto a lipophyllic surface will, in general, tend to be greater than the numbers of those molecules into the polar phase. The net effect can be described as a “partition coefficient”, which represents the relative amounts of a given solute in a 1 non-polar phase compared to the amount of that solute in a polar phase under equilibrium conditions. Thus, a molecule having a partition coefficient of greater than 1 is considered lipophyllic (or “hydrophobic”, or “non-polar”) and a molecule having a partition coefficient less than 1 is considered lipophobic (or “hydrophilic”or “polar”). Hydrophobic molecules at equilibrium, will be present in higher concentrations in non-polar phases than in polar phases.

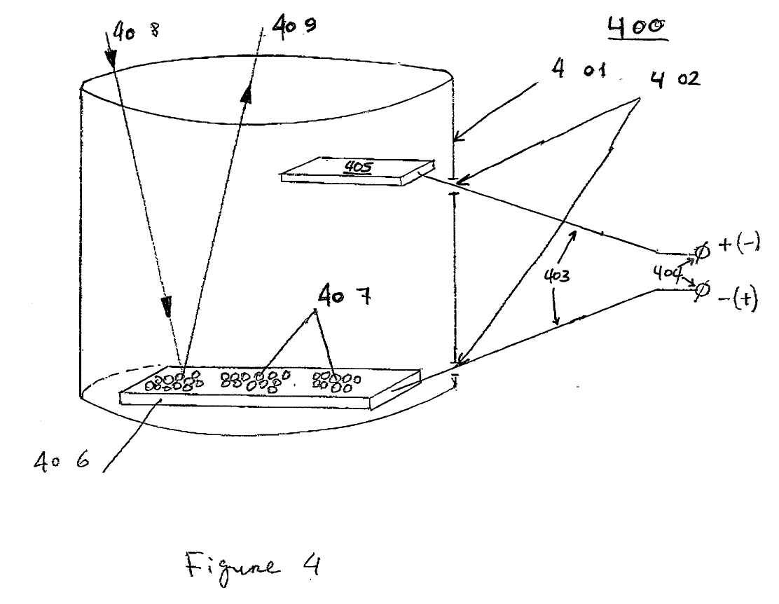

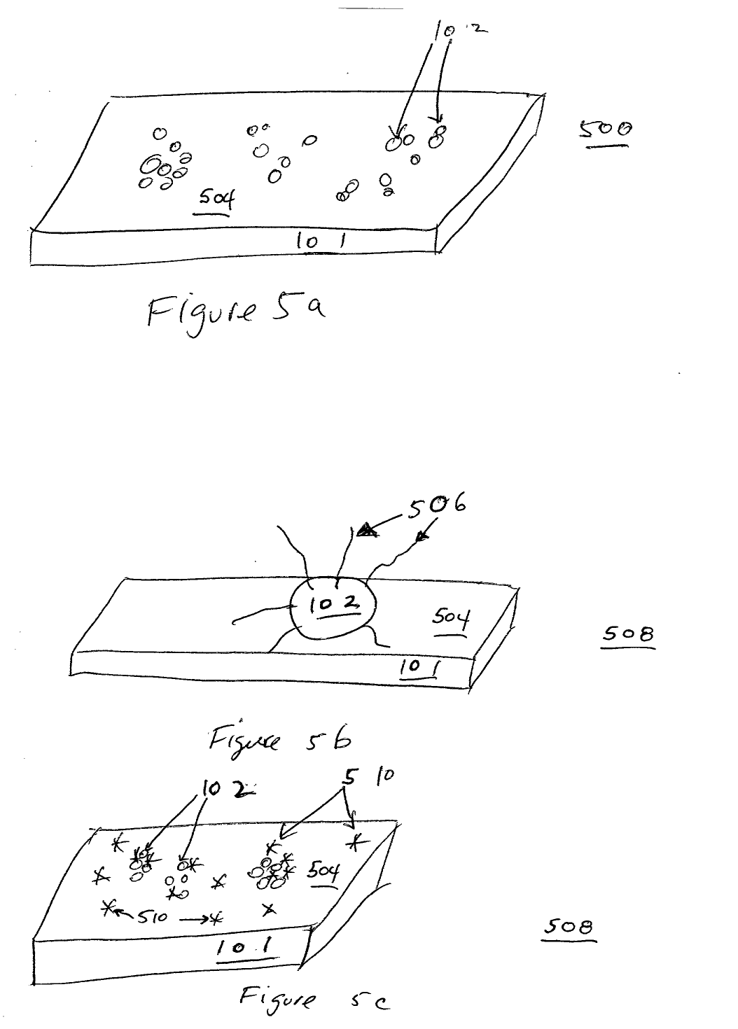

FIG. 5a depicts an alternative embodiment 500 of this invention wherein hydrophobic analytes are concentrated near enhancing structures 102 on substrate 504. Substrate 504 comprises a lipophyllic or hydrophobic substance. Enhancing structures 102 are made of gold, which, being relatively hydrophobic, can bind hydrophobic analytes. FIG. 5b depicts an alternative embodiment 508, wherein the enhancing structures 102 have additional hydrophobic moieties 506 attached thereto. FIG. 5c depicts an embodiment 508 of this invention after application of a hydrophobic analyte thereto. Upon application of a hydrophobic analyte 510 in a non-polar solvent (not shown), and upon evaporation of the solvent, the analyte 510 will tend to be distributed relatively evenly over the surface 504 and enhancing structures 102.

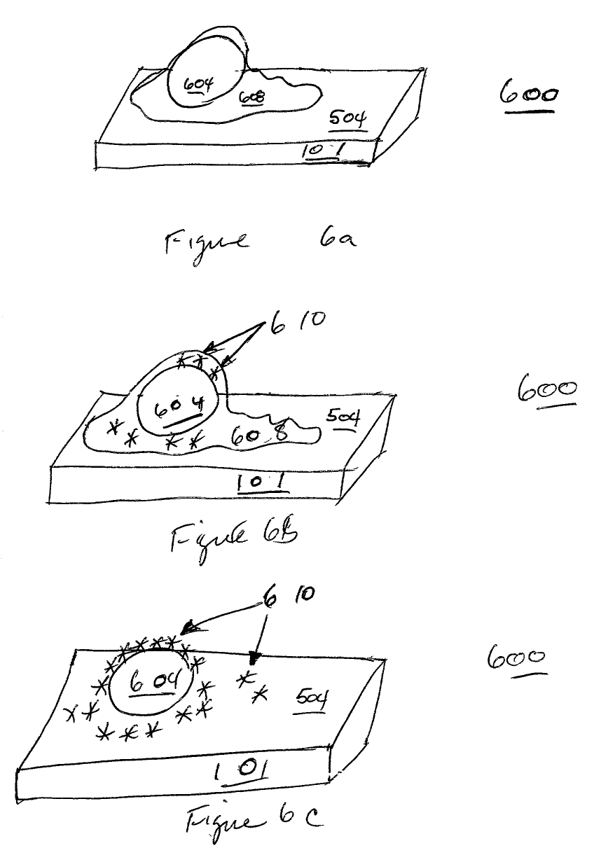

FIGS. 6a-6c depict an embodiment of this invention 600 wherein hydrophobic analytes are concentrated near enhancing structures. FIG. 6a depicts a substrate 101 having a hydrophilic surface 504. Hydrophobic enhancing structure 6004 is attached to surface 504. A liquid solvent 608 is applied to the surface 504 and the enhancing structure 604. The solvent 608 can come into contact with both surface 504 and enhancing structure 604. In FIG. 6b, analyte molecules 610 in solvent 608 are shown distributed throughout the solvent 608. FIG. 6c depicts the embodiments shown in FIGS. 6a and 6b after drying. Solvent 608 has evaporated, leaving analyte molecules 610 near surface 504 and enhancing structures 604.

The solvent can be either polar or non-polar. However, polar solvents will tend to wet polar enhancing structures and will therefore tend to concentrate solutes to the enhancing structures. Highly non-polar solvents will tend to wet the non-polar substrate and will tend not to wet the enhancing structures. Solvents of intermediate polarity can wet both enhancing structures and the substrate. Thus, it can be desirable to select solvent 608 to have non-polarity sufficient to wet the enhancing structure and thereby draw solute to the enhancing structures. Moreover, as the polarity of an enhancing structure is selected, one can select a solvent having polarity that provides a desired degree of wetting of the enhancing structures.

In alternative embodiments in which a polar solvent is relatively non-volatile, if the analyte partitions sufficiently onto the hydrophobic enhancing structures, the solvent can be withdrawn from the surface using capillary action, vacuum, blotting or other means known in the art, leaving the analyte near the enhancing structure.

Subsequently, if desired, the surface can be rinsed with additional solvent or another solvent to remove undesired materials from the enhancing structures. If the solvent is used that does not easily dissolve the analyte, such rinsing steps can be carried out without loss of the analyte, and can improve the sensitivity of detection.

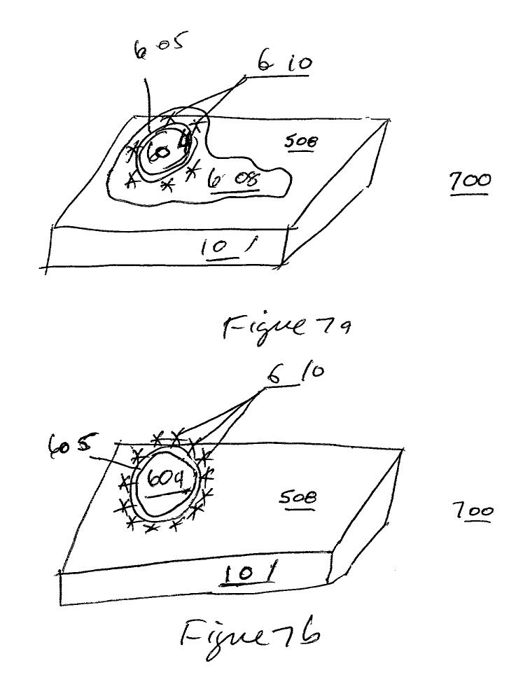

FIGS. 7a-7b depict yet another embodiment 700 of this invention in which hydrophillic analytes are concentrated near enhancing structures. Substrate 101 has hydrophobic surface 508 with hydrophilic enhancing structures 604 thereon. Enhancing structures 604 are made hydrophillic by the addition of hydrophilic material 605. Hydrophilic materials 605 can be alcohols, thiols, amines and other materials known in the art that can be attached to enhancing structures 604. Non-polar solvent 608 is applied to both substrate surface 508 and to enhancing structures 604. Analyte molecules 610 in the solvent 608 prefer to partition onto the hydrophilic enhancing structures 604, and not to prefer remain in solvent 608 or to partition to hydrophobic substrate 508. FIG. 7b depicts an embodiment as shown in FIG. 7a wherein solvent 608 has been removed by evaporation. Hydrophilic analytes 610 are shown near enhancing structures 604 and are not shown on hydrophobic surface 508.

In alternative embodiments, in which relatively non-volatile non-polar solvents are used, the solvent can be drawn off the substrate after the analyte has come into equilibrium with the enhancing structures. If the partition coefficient is selected properly, a substantial proportion of the analyte becomes partitioned onto the hydrophilic enhancing structures. Then, the non-polar solvent can be removed, leaving the hydrophilic analyte near or on the hydrophilic enhancing structures.

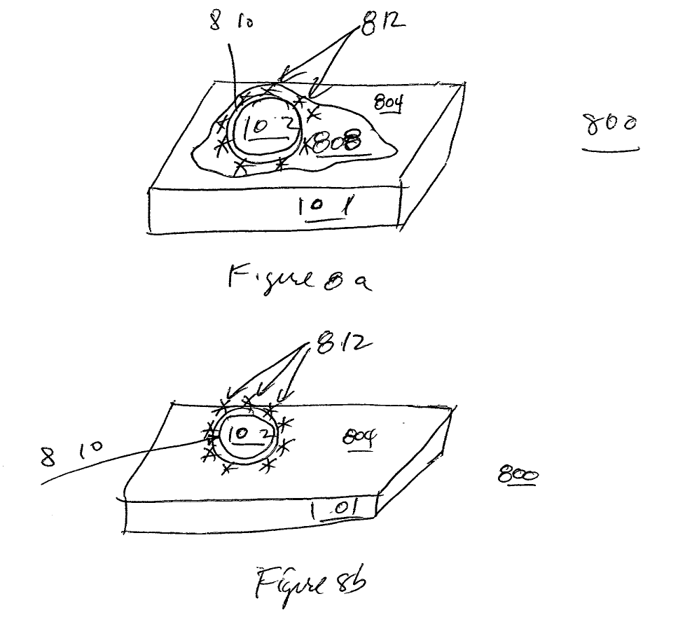

FIG. 8a-8b depict another embodiment 800 of this invention in which hydrophilic analytes are concentrated near hydrophilic enhancing structures. Surface 804 is hydrophobic. Enhancing structure 102 has a hydrophilic layer 810 thereon. Solvent 808 is hydrophilic. Polar analyte 812 is shown present in solvent 808. FIG. 8b depicts the embodiment 800 after evaporation of solvent 808, leaving hydrophilic analyte 812 preferentially concentrated near enhancing structure 102 and on hydrophilic layer 810.

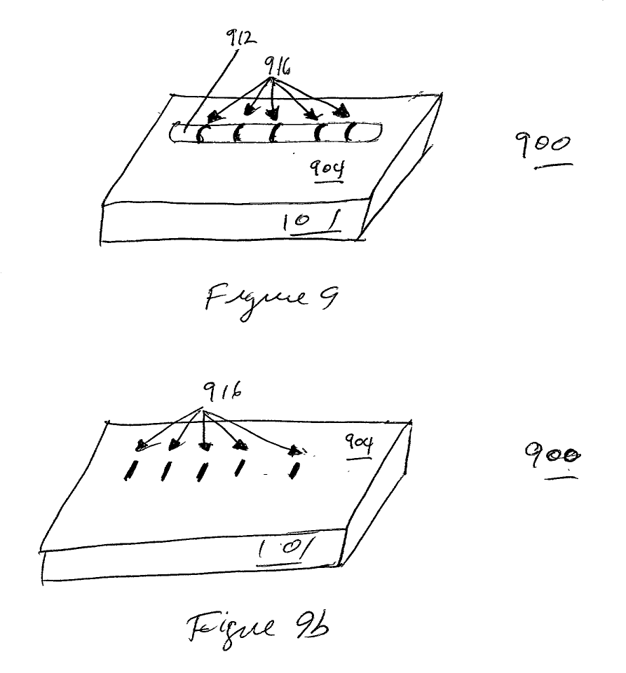

FIGS. 9a-9b depict another embodiment of this invention 900, in which analytes are subjected to isoelectric focusing in a gel and then transferred to a substrate for analysis. Isoelectric focusing gel 912 is shown after analytes 916 have been separated in an isoelectric focusing apparatus (not shown). Gel 912 is then placed on surface 904 which has enhancing structures thereon (not shown). Substrate 101 comprises surface 904 and enhancing structures (not shown). FIG. 9b depicts an embodiment as shown in FIG. 9a after transfer of analytes 916 to the surface 904. The transfer of analytes 916 to surface 904 can be accomplished using methods know in the art, such as drying, charge transfer and blotting.

It can be appreciated that in addition to isoelectric focusing, electrophoresis can be used to make an array of analytes. Such methods include capillary electrophoresis, two-dimensional electrophoresis and the like.

Additional embodiments include those in which a series of samples is collected on an array for storage, and then the array is placed on a surface having enhancing structures thereon. In optional embodiments, after transfer of the analytes to the substrate having enhancing structures, the substrate can be washed to remove analytes that are not affinity associated with enhancing structures.

Other embodiments of this invention include substrates onto which samples are placed for analysis. The surfaces can be prepared as described herein above but without enhancing structures being placed thereon. After a sample or series of samples has been adsorbed onto the surface, enhancing structures can be placed on top of the samples. Thus, analytes can be present near enhancing structures and can increase the sensitivity of analyte detection.

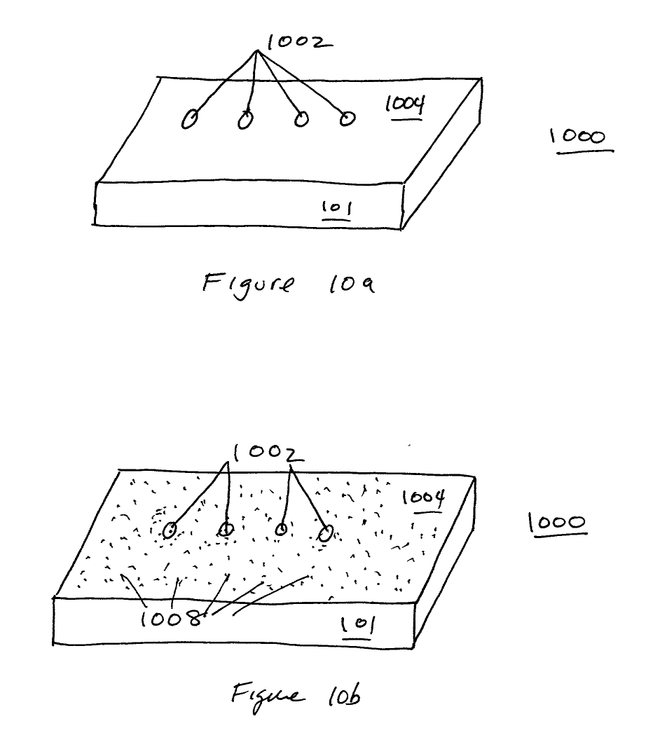

FIG. 10a depicts an embodiment 1000 of this invention in which analytes 1002 are placed on surface 1004. FIG. 10b depicts an embodiment of this invention as in FIG. 10a having enhancing structures 1008 placed thereon. Enhancing structures 1008 are near samples 1002, and enhance signals generated by samples 1002.

### EXAMPLE 4

### Detection of Silane on a Glass Substrate

To determine whether Raman signals can be observed for silane on a glass substrate, we treated the surface of a pre-cleaned glass microscope slide with isopropanol and then dried the slide. We found a Raman signal characteristic of glass. Then we placed a saturated solution of silane in isopropanol on the surface of the slide. Then, during drying, we stirred the surface of the sample with a pledget of paper. Upon completion of drying, the sample was throughly rinsed with isopropanol, followed by water, and finally again with isopropanol.

Upon drying, no Raman signal characteristic of silane was observed.

We then prepared a colloidal solution of silver fractal particles made as described herein above in Example 1, except that instead of gold, silver particles were generated. Upon addition of the colloidal solution, Raman signals characteristic of silane were prominent. Thus, we conclude that fractal structures and methods of this invention can enhance the detection of silane on a glass slide.

### EXAMPLE 5

### Detection of Silane on a Glass Substrate

We performed a similar series of experiments as described above for Example 4, except that we used a quartz slide instead of a glass slide. Unlike glass in Example 4, we found little Raman signal due to the quartz slide itself. However, upon application of silane in isopropanol, and treatment as described above for Example 4, we found a pronounced enhancement of the Raman signal characteristic of silane. Moreover, with the reduction in background Raman signal from the substrate, we conclude that quartz substrates can be useful for detecting analytes according to this invention.

### EXAMPLE 6

### Detection of Silane on an Aluminum Substrate

In another series of experiments, we measured Raman signals generated by silane on an aluminum foil surface. In the absence of silane, aluminum foil produces very little Raman signal. In the absence of enhancing structures, silane produced observable, but weak Raman signals. However, the Raman signals generated by silane were different from and greater than the signals generated by foil alone. Thus, silane can be detected by Raman spectroscopy in the absence of any added enhancing structures.

In other experiments, we prepared aluminum foil as we previously prepared glass and quartz slides with added enhancing structures (silver colloid fractals) as described in Examples 4 and 5 above. Under these conditions, the Raman signal produced by silane was increased substantially compared to the Raman signal generated by silane in the absence of enhancing structures.

The embodiments of this invention described herein are for illustration and are not intended to be limiting. Other embodiments incorporating the teachings of this invention can be readily appreciated by those of ordinary skill. All such modifications are included within the scope of this invention.

### INDUSTRIAL APPLICABILITY

Devices and methods are provided for detection of analytes using enhancing structures and means for localizing analytes near the enhancing structures. The devices and methods find use in industries in which detection and identification of analytes is of importance. The devices and methods find use in biological sciences for diagnosis of physiological and pathophysiological conditions.

We claim:

1. A device for analyte detection, comprising:
a substrate having a surface thereon;
an area of said surface having at least one enhancing structure thereon; and
means for localizing an analyte near said enhancing structure.

2. A device for analyte detection, comprising:
a porous substrate having a surface thereon; and
an area having at least one enhancing structure on said surface.

3. The device of claim 2, wherein said porous substrate has pores sufficiently small to retain analyte molecules on said surface, and permitting solvent molecules to pass through said substrate.

4. The device of claim 3, wherein said substrate is within a spin-concentrating device, said substrate dividing said spin-concentrating device into an upper portion and a lower portion.

5. The device of claim 4, further comprising means for holding said device in a centrifuge.

6. The device of claim 4, further comprising means for providing a pressure gradient from said upper portion of said cylinder to said lower portion of said cylinder.

7. A device for detecting a hydrophilic analyte, comprising:
a substrate having a hydrophobic surface thereon; and
an area having at least one hydrophilic enhancing structure on said surface.

8. A device for detecting a hydrophobic analyte, comprising:
a substrate having a hydrophilic surface thereon; and
an area having at least one enhancing structure on said surface, said analyte having hydrophobicity sufficient to localize said analyte near said at least one enhancing structure.

9. A method for detecting an analyte, comprising the steps of:
(a) providing a device comprising:
a substrate having a surface thereon;
an area of said surface having at least one enhancing structure thereon; and
means for localizing an analyte near said enhancing structure;
(b) placing a sample comprising an analyte on said surface;
(c) permitting said analyte to localize near said at least one enhancing structure; and
(d) detecting the presence of said analyte near said at least one enhancing structure.

10. A method for detecting an analyte, comprising the steps of:
(a) providing a device comprising:
a substrate having a porous surface having pores therein sufficiently small to retain analyte molecules on said surface and permitting solvent molecules to pass through; and
an area having at least one enhancing structure on said surface;
(b) applying a sample comprising an analyte on said surface;
(c) moving said solvent through said pores, thereby concentrating said analyte molecules near said enhancing structures; and
(d) detecting the presence of said analyte near said at least one enhancing structure.

11. A method for detecting an analyte, comprising the steps of:
(a) providing a device comprising:
a cylinder comprising a substrate having a porous surface having pores therein sufficiently small to retain analyte molecules on said surface and permitting solvent molecules to pass through, said substrate dividing said cylinder into an upper portion and a lower portion; and
an area having at least one enhancing structure on said surface;
(b) applying a sample comprising an analyte on said surface;
(c) moving said solvent through said pores, thereby concentrating said analyte molecules near said enhancing structures; and
(d) detecting the presence of said analyte near said at least one enhancing structure.

12. The method of claim 11, wherein said step of moving is carried out using a pressure gradient.

13. The method of claim 12, wherein said pressure gradient is provided by decreasing hydrostatic pressure in said lower portion of said cylinder.

14. The method of claim 12, wherein said pressure gradient is provided by increasing hydrostatic pressure in said upper portion of said cylinder.

15. The method of claim 11, wherein said step of moving is carried out using a centrifuge.

16. A method for detecting a hydrophilic analyte, comprising the steps of:
(a) providing a device comprising:
a substrate having a hydrophobic surface thereon; and
an area having at least one enhancing structure on said surface;
(b) applying a sample comprising an analyte on said surface, thereby permitting said analyte to localize near said at least one enhancing structure; and
(c) detecting the presence of said analyte near said at least one enhancing structure.

17. The device of claim 1, wherein said at least one enhancing structure comprises a fractal particle associate.

18. The device of claim 17, wherein said fractal particle associate comprises gold or silver particles.

19. The device of claim 2, wherein said at least one enhancing structure is a fractal particle associate.

20. The device of claim 19, wherein said fractal particle associate comprises gold or silver particles.

21. The method of claim 9, wherein said step of permitting includes permitting solvent evaporation to occur.

22. A device for analyte detection, comprising:
a first electrode having at least one enhancing structure thereon, said first electrode within an analysis chamber; and
a second electrode.

23. A method for detecting a charged analyte, comprising the steps of:
(a) providing a device comprising:
a first electrode having at least one enhancing structure thereon; and
a second electrode, wherein at least said first electrode is within an analysis chamber;
(b) forming a potential difference between said first and said second electrodes;
(c) applying a sample having a charged analyte therein into said analysis chamber;
(d) permitting said charged analyte to localize to said first electrode; and
(e) detecting the presence of said charged analyte near said at least one enhancing structure on said first electrode.

24. The device of claim 22, wherein said second electrode is within an said analysis chamber and has at least one enhancing structure thereon.

25. A method for detecting an analyte in a mixture of molecules, comprising the steps of:
(a) providing a substrate having a plurality of enhancing structures thereon;
(b) applying to said substrate an spatial array of molecules containing said analyte; and
(c) detecting the presence of said analyte near at least one of said plurality of enhancing structures.

26. The method of claim 25, wherein said spatial array is provided by capillary electrophoresis.

27. The method of claim 25, wherein said spatial array is provided by isoelectric focusing.

28. The device of claim 1, wherein said substrate has a metal layer thereon.

29. The device of claim 2, wherein said substrate has a metal layer thereon.
